(19) United States
(12) Patent Application Publication
(10) Pub. No.: US 2023/0132199 A1
(43) Pub. Date: Apr. 27, 2023

(54) METHODS AND SYSTEMS FOR PROCESSING SAMPLES

(71) Applicant: IDBYDNA INC., San Francisco, CA (US)

(72) Inventors: Hajime MATSUZAKI, Cupertino, CA (US); Guochun LIAO, Belmont, CA (US); Yuying MEI, Sunnyvale, CA (US)

(21) Appl. No.: 17/259,518

(22) PCT Filed: Jul. 11, 2019
(86) PCT No.: PCT/US2019/041447

§ 371 Date: Jan. 11, 2021

Related U.S. Application Data
(60) Provisional application No. 62/696,783, filed on Jul. 11, 2018.

(51) Int. Cl. G16B 30/10; C12Q 1/6869; G16B 20/20

(52) U.S. Cl. CPC G16B 30/10; C12Q 1/6869; G16B 20/20

(57) ABSTRACT

The present disclosure provides methods and systems for processing samples including nucleic acid molecules. The methods may comprise identifying polymorphisms in a plurality of sequencing libraries and using the polymorphisms to identify the plurality of sequencing libraries as being associated with the same sample.

### CROSS-REFERENCE

This application claims the benefit of U.S. Provisional Patent Application No. 62/696,783 filed Jul. 11, 2018, which is entirely incorporated herein by reference.

### BACKGROUND

Samples may be analyzed for various purposes, including detecting the presence or amount of a target such as a nucleic acid molecule in a sample. Analysis of a sample comprising one or more nucleic acid molecules may involve sequencing the nucleic acid molecules, or portions or derivatives thereof. Sequencing may facilitate identification of contaminants and/or species of potential interest within a sample. For example, sequencing may be used to identify a microorganism or pathogen within a sample.

### SUMMARY

Recognized herein is a need to improve diagnostic testing for pathogens in patient samples. A diagnostic test may involve extracting ribonucleic acid (RNA) and deoxyribonucleic acid (DNA) molecules from a patient sample and preparing (e.g., independently preparing) sequencing libraries for both the RNA (e.g., RNA converted to complementary DNA (cDNA)) and DNA molecules. In addition to representing microorganisms, which may include pathogens and the normal microbiota present in the sample, these sequencing libraries contain the patients' Human sequences. A plurality of samples may be analyzed using the same instrumentation, simultaneously, and/or in close proximity to one another. Although highly trained technologists perform the library preparation in accordance with standard operating procedures that are designed to assure correct sample and library identity, there is always a slight possibility of sample mis-assignment, where the RNA library is not from the same patient sample as the DNA library.

Accordingly, the present disclosure provides methods and systems for processing and identifying samples including nucleic acid molecules or derivatives thereof (e.g., sequencing reads). A sample comprising a plurality of RNA molecules and a plurality of DNA molecules may be separately processed to provide an RNA sequencing library and a DNA sequencing library. A marker that is shared between the RNA and DNA libraries may be identified and used to identify the libraries as deriving from the same patient sample. For example, polymorphisms in the Human sequences may be genotyped and then matched. Two readily applicable categories of Human polymorphisms are 1) single nucleotide polymorphisms (SNPs), and 2) haplogroups in the mitochondrial DNA (mtDNA). In the case of SNPs, a small subset of about one hundred loci that are in expressed regions and highly polymorphic across a diversity of ethnicities may be selected for genotyping. This approach is similar to subsets of polymorphic SNPs, referred to as Ancestry Informative Markers (AIMs), that may be used in a variety of genomic applications, from anthropology to stratifying case-control association studies for Human diseases. Similarly, mtDNA genotyping which results in identifying haplogroups, may be used to study Human diversity and global migration.

In an aspect, the present disclosure provides a method of identifying a polymorphism, comprising (a) providing a ribonucleic acid (RNA) sequencing library and a deoxyribonucleic acid (DNA) sequencing library, wherein the RNA sequencing library and the DNA sequencing library derive from the same sample; (b) identifying one or more polymorphisms in the RNA sequencing library and one or more polymorphisms in the DNA sequencing library; and (c) identifying a polymorphism of the RNA sequencing library and a polymorphism of the DNA sequencing library as being the same.

In some embodiments, the method further comprises, prior to (c), assigning each polymorphism of the one or more polymorphisms of the RNA sequencing library and the one or more polymorphisms of the DNA sequencing library a random index, wherein the random index assigned to a given polymorphism for the RNA sequencing library is the same as the random index assigned to the given polymorphism for the DNA sequencing library. In some embodiments, the random index comprises hashes, numbers and/or integers.

In some embodiments, the one or more polymorphisms of the RNA sequencing library and the one or more polymorphisms of the DNA sequencing library are selected from the group consisting of single nucleotide polymorphisms and haplogroups. In some embodiments, the one or more polymorphisms of the RNA sequencing library and the one or more polymorphisms of the DNA sequencing library are single nucleotide polymorphisms.

In some embodiments, the method may further comprise generating the RNA sequencing library and the DNA sequencing library. In some embodiments, generating the RNA sequencing library comprises providing a sample comprising a plurality of RNA molecules and a plurality of DNA molecules. In some embodiments, the plurality of RNA molecules and the plurality of DNA molecules are separated. In some embodiments, the RNA sequencing library and the DNA sequencing library are prepared simultaneously. In some embodiments, generating the RNA sequencing library and/or the DNA sequencing library comprises sequencing by synthesis or nanopore sequencing. In some embodiments, generating the RNA sequencing library comprises reverse transcribing the plurality of RNA molecules.

In some embodiments, the sample comprises one or more cells. In some embodiments, the method further comprises lysing the one or more cells.

In some embodiments, the RNA sequencing library and the DNA sequencing library are derived from a bodily fluid. In some embodiments, the bodily fluid is selected from the group consisting of blood, urine, saliva, and sweat.

In some embodiments, the sample derives from a patient. In some embodiments, the patient has or is suspected of having a disease or disorder. In some embodiments, the patient has been exposed or is suspected of having been exposed to a pathogen.

In another aspect, the present disclosure provides a method identifying a polymorphism, comprising: (a) providing a ribonucleic acid (RNA) sequencing library and a deoxyribonucleic acid (DNA) sequencing library, wherein the RNA sequencing library and the DNA sequencing library derive from the same sample; (b) identifying one or more polymorphisms of the RNA sequencing library and one or more polymorphisms of the DNA sequencing library; (c) obfuscating the one or more polymorphisms in the RNA sequencing library and the one or more polymorphisms in the DNA sequencing library; and (d) identifying a polymorphism of the RNA sequencing library and a polymorphism of the DNA sequencing library as being the same.

In some embodiments, based on (d), the RNA sequencing library and the DNA sequencing library are identified as deriving from the same sample.

In some embodiments, the method further comprises, prior to (c), assigning each polymorphism of the one or more polymorphisms of the RNA sequencing library and the one or more polymorphisms of the DNA sequencing library a random index, wherein the random index assigned to a given polymorphism for the RNA sequencing library is the same as the random index assigned to the given polymorphism for the DNA sequencing library. In some embodiments, the random index comprises hashes, numbers and/or integers.

In some embodiments, the one or more polymorphisms of the RNA sequencing library and the one or more polymorphisms of the DNA sequencing library are selected from the group consisting of single nucleotide polymorphisms and haplogroups. In some embodiments, the one or more polymorphisms of the RNA sequencing library and the one or more polymorphisms of the DNA sequencing library are single nucleotide polymorphisms.

In some embodiments, the method may further comprise generating the RNA sequencing library and the DNA sequencing library. In some embodiments, generating the RNA sequencing library comprises providing a sample comprising a plurality of RNA molecules and a plurality of DNA molecules. In some embodiments, the plurality of RNA molecules and the plurality of DNA molecules are separated. In some embodiments, the RNA sequencing library and the DNA sequencing library are prepared simultaneously. In some embodiments, generating the RNA sequencing library and/or the DNA sequencing library comprises sequencing by synthesis or nanopore sequencing. In some embodiments, generating the RNA sequencing library comprises reverse transcribing the plurality of RNA molecules.

In some embodiments, the sample comprises one or more cells. In some embodiments, the method further comprises lysing the one or more cells.

In some embodiments, the RNA sequencing library and the DNA sequencing library are derived from a bodily fluid. In some embodiments, the bodily fluid is selected from the group consisting of blood, urine, saliva, and sweat.

In some embodiments, the sample derives from a patient. In some embodiments, the patient has or is suspected of having a disease or disorder. In some embodiments, the patient has been exposed or is suspected of having been exposed to a pathogen.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a sample workflow in which materials are correctly associated with the same patient;

FIG. 2 shows a sample workflow in which materials are incorrectly associated with the same patient; and

FIG. 3 shows a computer system that is programmed or otherwise configured to implement methods of the present disclosure herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The present disclosure provides methods of identifying polymorphisms in sequencing libraries. The methods may comprise providing a plurality of sequencing libraries (e.g., an RNA sequencing library and a DNA sequencing library) associated with a sample, identifying one or more polymorphisms in the plurality of sequencing libraries, and identifying a polymorphism associated with a first sequencing library of the plurality of sequencing libraries and a polymorphism associated with a second sequencing library of the plurality of sequencing libraries as being the same. In some cases, identifying the polymorphisms as being the same may identify the sequencing libraries with which they are associated as deriving from the same sample, such as from the same sample from a patient.

A plurality of sequencing libraries may be associated with the same sample. A sample may derive from a patient (e.g., a human patient). A patient from which a sample derives may have or be suspected of having a disease or disorder. In some cases, a patient from which a sample derives may have or be suspected of having a disease or disorder associated with a pathogen (e.g., bacteria, fungi, or virus). In some cases, a patient from which a sample derives may have been exposed or be suspected of having been exposed to a pathogen.

A sample may comprise a bodily fluid, such as blood, urine, saliva, or sweat. A sample may comprise one or more cells, and/or may comprise cell-free nucleic acid molecules. Cells of a sample may be lysed to provide access to a plurality of nucleic acid molecules therein.

Sequencing libraries may be provided for analysis and processing. Sequencing libraries may be generated from a plurality of nucleic acid molecules (e.g., a plurality of RNA molecules and a plurality of DNA molecules) of a sample (e.g., a sample from a patient). Generating a sequencing library may comprise sequencing by synthesis, nanopore sequencing, sequencing by ligation, sequencing by hybridization, or another method. In some cases, generating a sequencing library may comprise next generation sequencing (NGS) using, for example, the Illumina NGS platform. Sequencing libraries for different populations of nucleic acid molecules may be generated separately and/or simultaneously. For example, a DNA sequencing library and an RNA sequencing library may be prepared separately. Generating an RNA sequencing library may comprise reverse transcribing a plurality of RNA molecules to provide a plurality of complementary DNA (cDNA) molecules). Sequencing reads may be provided in, for example, fastq file format.

Polymorphisms such as single nucleotide polymorphisms (SNPs) and mitochondrial deoxyribonucleic acid (mtDNA) haplogroups may be detected in sequencing data (e.g., data produced using next-generation sequencing, such as from the Illumina platform) by aligning sequencing reads to a reference and applying a probabilistic model. For SNPs, the reference may be a Human genome build, while the reference for mtDNA may be a Reconstructed Sapiens Reference Sequence (RSRS). SNP genotyping may comprise the use of a software application such as GATK or FreeBayes. The same or different software may be used to identify mtDNA haplogroups. In some cases, identifying mtDNA haplogroups may comprise the use of a software application such as MToolBox or mitoMap.

Determining SNP genotypes and mtDNA haplogroups may indirectly expose patients' protected health information (PHI). Certain SNP loci may be indicative of Human diseases through linkage disequilibrium, which is the underlying basis for case-control association studies. The polymorphisms used to determine the mtDNA haplogroup may be associated with mitochondrial diseases. Although in practice such associations with diseases are likely to be very rare, the SNP genotyping and mtDNA haplogroups can reveal the ethnicity of a patient, as well as, the ethnicity of the patient's mother. To circumvent this unnecessary exposure to PHI, the SNP genotypes and mtDNA haplogroups will be obfuscated. The accuracy of the genotypes and haplogroups may not be necessary; most important for this application would be that the polymorphisms are detected with the required precision to match the RNA and DNA sequencing libraries.

The obfuscation of SNP genotypes and mtDNA haplogroups may rely on the use of random hashes. For SNPs, a hash table may assign a random index (such as a unique integer) to each of the hundred or so loci. The genome positions of the loci may be hidden; and the random index insures that genotypes may be output in a different order for every patient sample. For mtDNA haplogroups, the clades in the mitochondrial phylogenic tree are denoted by alphabet and the sub-clades by an integer, for example, C4; the hash table may re-assign a random unique letter to the clades, and a random unique integer to the subsequent sub-clade. The lower levels of haplogroup, such as the “a1” in C4a1 may also be re-assigned with letters and integers. Since both the RNA and DNA libraries may use the same hash, the depth of the haplogroup (branches in tree) may be preserved in the comparison between haplogroup calls.

In some cases, the comparison of SNP genotypes between the libraries may be complicated by heterozygous genotypes. For a variety of reasons, such as allele specific expression or low read coverage, a true heterozygous genotype may be mis-called as homozygous. A probability model that accounts for the frequencies of this type of mis-calling could be developed to measure the confidence of a match between sets of genotype calls at the hundred or so selected SNPs. Data (e.g., existing data) from, for example, RNASeq could be used to select SNPs in expressed regions, and compared with genotypes from, for example, DNASeq data to help build the model.

In some cases, the comparison of mtDNA haplogroup may be complicated by differences in the depths of the haplogroup call between the RNA and DNA libraries. If read coverage is low, the haplogroup call is likely to be shallow (closer to the major clades). Like expressed SNP sites, read coverage in the RNASeq is dependent on expression levels in the patients' mitochondria; and, the read coverage from DNASeq may vary due to variations in the DNA extraction and Human depletion process. Data (e.g., existing data) to various low read coverages can help create a model that relates haplogroup call depth and true library matches.

In some cases, a patient sample may be analyzed more than one time. For example, a user may wish to verify a result of an analysis, particularly if a first analysis did not satisfy all quality control criteria for a sequencing process and/or sample library preparation. The same approach of using a Human polymorphism to match RNA and DNA sequencing libraries within an analysis may also be used to match libraries across experiments (e.g., runs) when the same patient sample is re-analyzed in a subsequent experiment.

The process of aligning reads to reference sequences in current methods, such as GATK and MToolBox, may be highly time consuming. Instead, Taxonomer software (Flygare 2016, DOI:10.1186/s13059-016-0969-1) enables highly computationally efficient sequence comparisons by decomposing reads into multiple k-mers which can be matched to indexed k-mers derived from reference databases of known sequences. The Binner component of Taxonomer software can be used to rapidly segregate sequencing reads that correspond to SNP loci of interest and to the mtDNA. To reduce bias at SNPs, the Binner references could contain all known alleles of the one hundred or so selected polymorphisms. The allele balanced Binner references can be extensively tested by using publicly available data from the 1000 Genomes Project, which contains NGS Illumina platform data from Human individuals representing a variety of ethnicities. Similarly, to reduce bias in the mtDNA, all >15,000 records of Human mitochondrial genomes in GenBank can be used as Binner references. The use of Taxonomer Binner software may greatly reduce the computational analysis times in the search for Human polymorphisms. will be highly complementary to the main search for pathogens.

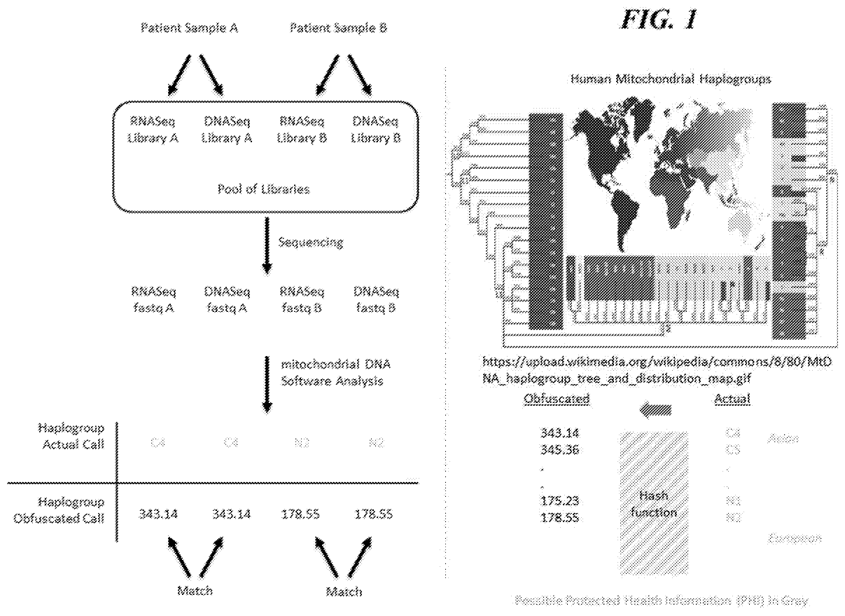

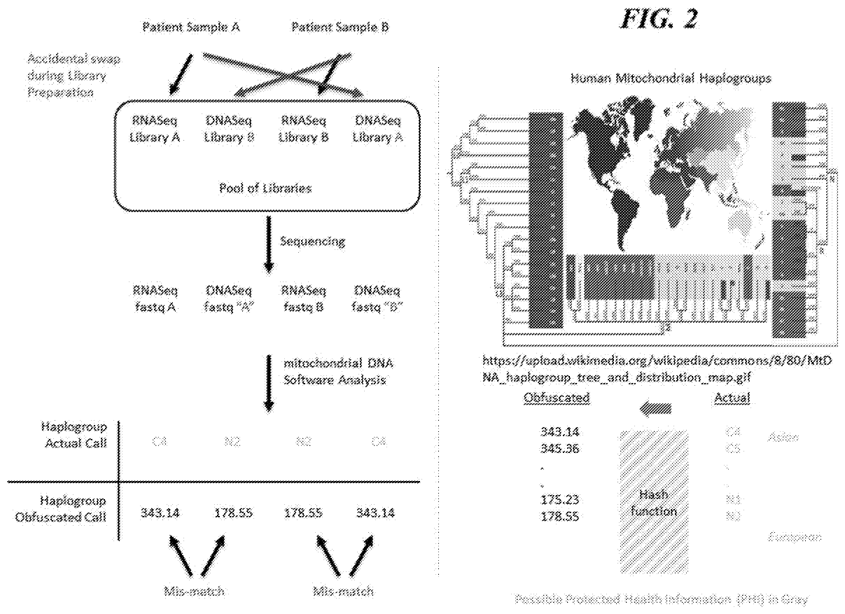

FIG. 1 shows a sample workflow in which materials are correctly associated with the same patient, while FIG. 2 shows a sample workflow in which materials are incorrectly associated with the same patient. In each figure, the left panel includes a flow chart of processing and sequencing two hypothetical patient samples and the right panel shows mitochondrial haplogroups and how a hash function can be used to obfuscate the haplogroup calls, which may be associated with protected health information (PHI) as they may inform ancestry.

### Computer Systems

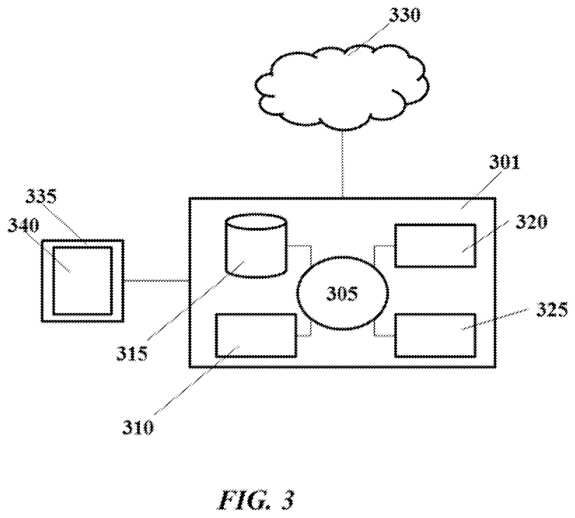

The present disclosure provides computer systems that are programmed to implement methods of the disclosure. FIG. 3 shows a computer system 301 that is programmed or otherwise configured to process and/or assay a sample. The computer system 301 may regulate various aspects of sample processing and assaying of the present disclosure, such as, for example, activation of a valve or pump to transfer a reagent or sample from one chamber to another or application of heat to a sample (e.g., during an amplification reaction). The computer system 301 may be an electronic device of a user or a computer system that is remotely located with respect to the electronic device. The electronic device may be a mobile electronic device.

The computer system 301 includes a central processing unit (CPU, also “processor” and “computer processor” herein) 305, which may be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 may be a data storage unit (or data repository) for storing data. The computer system 301 may be operatively coupled to a computer network (“network”) 330 with the aid of the communication interface 320. The network 330 may be the Internet, an internet and/or extranet, or an intranet and/or extranet that is in communication with the Internet. The network 330 in some cases is a telecommunication and/or data network. The network 330 may include one or more computer servers, which may enable distributed computing, such as cloud computing. The network 330, in some cases with the aid of the computer system 301, may implement a peer-to-peer network, which may enable devices coupled to the computer system 301 to behave as a client or a server.

The CPU 305 may execute a sequence of machine-readable instructions, which may be embodied in a program or software. The instructions may be stored in a memory location, such as the memory 310. The instructions may be directed to the CPU 305, which may subsequently program or otherwise configure the CPU 305 to implement methods of the present disclosure. Examples of operations performed by the CPU 305 may include fetch, decode, execute, and writeback.

The CPU 305 may be part of a circuit, such as an integrated circuit. One or more other components of the system 301 may be included in the circuit. In some cases, the circuit is an application specific integrated circuit (ASIC).

The storage unit 315 may store files, such as drivers, libraries and saved programs. The storage unit 315 may store user data, e.g., user preferences and user programs. The computer system 301 in some cases may include one or more additional data storage units that are external to the computer system 301, such as located on a remote server that is in communication with the computer system 301 through an intranet or the Internet.

The computer system 301 may communicate with one or more remote computer systems through the network 330. For instance, the computer system 301 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 301 via the network 330.

Methods as described herein may be implemented by way of machine (e.g., computer processor) executable code stored on an electronic storage location of the computer system 301, such as, for example, on the memory 310 or electronic storage unit 315. The machine executable or machine readable code may be provided in the form of software. During use, the code may be executed by the processor 305. In some cases, the code may be retrieved from the storage unit 315 and stored on the memory 310 for ready access by the processor 305. In some situations, the electronic storage unit 315 may be precluded, and machine-executable instructions are stored on memory 310.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 301, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 may include or be in communication with an electronic display 335 that comprises a user interface (UI) 340 for providing, for example, a current stage of processing or assaying of a sample (e.g., a particular operation, such as a lysis operation, that is being performed). Examples of UI's include, without limitation, a graphical user interface (GUI) and web-based user interface.

Methods and systems of the present disclosure may be implemented by way of one or more algorithms. An algorithm may be implemented by way of software upon execution by the central processing unit 305.

### EXAMPLES

### Example 1

### Proof of Concept

Three sequencing libraries were prepared from a patient sample. Two of the libraries were RNA, and tested the effect of using Ribo Zero to deplete ribosomal RNA; the third library was a DNA library. The libraries were sequenced on an Illumina MiSeq; and fastq data were processed in MToolBox to determine mtDNA haplogroups. The results are summarized in the table below.

| | | mtDNA | Per base | Best predicted |
|---|---|---|---|---|
| | Sample | Coverage | depth | haplogroup(s) |
| RNASeq | H1-20160610-RZ_S4_L001_R1_001 | 94.8 | 67.1 | C4a1d |
| RNASeq | H1-20160610-nonRZ_S1_L001_R1_001 | 98.6 | 1394.7 | C4a1d |
| DNASeq | H1-20160610-DNA_S1_L001_R1_001 | 100.0 | 285.7 | C4a1d |

The mtDNA haplogroup calls are consistent among the three libraries, strongly confirming that they are derived the same patient sample. Here, the haplogroup calls are not obfuscated. Note: the Ribo Zero (first RNA library “RZ”) appears to lower mitochondrial transcripts in addition to depleting ribosomal RNA.

Several aspects are described with reference to example applications for illustration. Unless otherwise indicated, any embodiment may be combined with any other embodiment. It should be understood that numerous specific details, relationships, and methods are set forth to provide a full understanding of the features described herein. A skilled artisan, however, will readily recognize that the features described herein may be practiced without one or more of the specific details or with other methods. The features described herein are not limited by the illustrated ordering of acts or events, as some acts may occur in different orders and/or concurrently with other acts or events. Furthermore, not all illustrated acts or events are required to implement a methodology in accordance with the features described herein.

Some inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Alternatively, particularly with respect to biological systems or processes, the term may mean within an order of magnitude, within 5-fold, or within 2-fold, of a value. Where particular values are described in the application and claims, unless otherwise stated the term “about” meaning within an acceptable error range for the particular value may be assumed.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method of associating a plurality of sequencing libraries with the same human patient, comprising:
(a) providing a plurality of sequencing libraries generated from different populations of nucleic acid molecules of a first sample, in a plurality of samples, that are analyzed using the same instrumentation in close proximity to one another, wherein the first sample is from a first human patient and the nucleic acid molecules of the first sample include nucleic sequences from the first human patient and nucleic acid sequences from one or more microorganisms;
(b) identifying one or more polymorphisms from the human nucleic sequences in each sequencing library in the plurality of sequencing libraries; and
(c) using the one or more polymorphisms in each sequencing library in the plurality of sequence libraries to correctly associate the plurality of sequence libraries with the first human patient.

2. The method of claim 1, wherein a first sequencing library in the plurality of sequencing libraries is an RNA sequencing library and a second sequencing library in the plurality of sequencing libraries is a DNA sequencing library, the method further comprising, prior to (c), obfuscating each respective polymorphism of the one or more polymorphisms of the RNA sequencing library and the one or more polymorphisms of the DNA sequencing library with a corresponding obfuscating index, wherein the corresponding obfuscating index assigned to a given polymorphism for the RNA sequencing library is the same as the corresponding obfuscating index assigned to the given polymorphism for the DNA sequencing library.

3. The method of claim 2, wherein the corresponding obfuscating index is a random index that comprises hashes, numbers and/or integers.

4. The method of claim 2, wherein the one or more polymorphisms of the RNA sequencing library and the one or more polymorphisms of the DNA sequencing library are selected from the group consisting of single nucleotide polymorphisms and haplogroups.

5. The method of claim 4, wherein the one or more polymorphisms of the RNA sequencing library and the one or more polymorphisms of the DNA sequencing library are single nucleotide polymorphisms.

6. The method of any one of claims 2-5, further comprising generating the RNA sequencing library from a population of RNA molecules in the first sample and generating the DNA sequencing library from a population of DNA molecules in the first sample.

7. (canceled)

8. The method of claim 6, wherein the plurality of RNA molecules and the plurality of DNA molecules are separated.

9. The method of claim 6, wherein the RNA sequencing library and the DNA sequencing library are prepared separately or simultaneously.

10. The method of claim 6, wherein generating the RNA sequencing library and/or the DNA sequencing library comprises sequencing by synthesis or nanopore sequencing.

11. The method of claim 6, wherein generating the RNA sequencing library comprises reverse transcribing a plurality of RNA molecules.

12. The method of claim 1, wherein the first sample comprises one or more cells and the method further comprises lysing the one or more cells.

13. (canceled)

14. The method of claim 1, wherein the RNA first sample is a bodily fluid.

15. The method of claim 14, wherein the bodily fluid is selected from the group consisting of blood, urine, saliva, and sweat.

16. The method of claim 1, wherein the sample derives from the first human patient.

17. The method of claim 16, wherein the first human patient has or is suspected of having a disease or disorder.

18. The method of claim 16, wherein the first human patient has been exposed or is suspected of having been exposed to a pathogen.

19. The method of claim 2, wherein:
the using the one or more polymorphisms in each sequencing library in the plurality of sequence libraries to correctly associate the plurality of sequence libraries with the first human patient uses the corresponding obfuscated indexes of the one or more polymorphisms of the RNA sequencing library and the corresponding obfuscated indexes of the one or more polymorphisms from the DNA sequencing library to correctly associate the plurality of sequence libraries with the first patient.

20-37. (canceled)

38. The method of claim 1, wherein the one or more polymorphisms is a mitochondrial deoxyribonucleic acid (mtDNA) haplogroup.

39. The method of claim 1, wherein the one or more polymorphisms is a SNP genotype.

40. The method of claim 1, wherein the one or more microorganisms are pathogens.

41. The method of claim 1, wherein the method further comprises using the plurality of sequencing libraries to identify the one or more microorganisms.

42. The method of claim 6 wherein the RNA sequencing library and the DNA sequencing library are prepared separately.

43. The method of claim 1, the method further comprising:
obfuscating the one or more polymorphisms in the RNA sequencing library and the one or more polymorphisms in the DNA sequencing library; and
the using the one or more polymorphisms in each sequencing library in the plurality of sequence libraries to correctly associate the plurality of sequence libraries with the first human patient uses the one or more polymorphisms of the RNA sequencing library in obfuscated form and the one or more polymorphisms from the DNA sequencing library in obfuscated form to correctly associate the plurality of sequence libraries with the first patient.
